(19) United States
(12) Patent Application Publication
(10) Pub. No.: US 2002/0173750 A1
(43) Pub. Date: Nov. 21, 2002

(54) Syringe device

(76) Inventors: Chiu-Yueh Huang, Taipei City (TW); Shih-Ching Lin, Taichung City (TW)

Correspondence Address: Charles E. Baxley, Hart, Baxley, Daniels & Holton, Fifth Floor, 59 John Street, New York, NY, 10038, US

(21) Appl. No.: 09/859,303

(22) Filed: May 18, 2001

(51) Int. Cl. A61M 5/00

(52) U.S. Cl. 604/218

(57) ABSTRACT

A syringe device includes a core member received in the plunger and the core member is fixedly connected to a base member to which a needle cannula is connected. When the plunger is pulled, the base member is pulled into the barrel. Two flexible protrusions extend from the plunger and which expand wide when the plunger is pulled to a desired position. The expanded flexible protrusions limit the plunger from being pushed into the barrel again.

### FIELD OF THE INVENTION

The present invention relates to a syringe device wherein the needle cannula is retracted into the barrel by pulling the plunger after the medicine in the tube is injected, and the plunger cannot be pushed into the barrel.

### BACKGROUND OF THE INVENTION

A conventional syringe device is shown in FIG. 1 and generally includes a barrel 10 with an engaging tube 12 extending from an end of the barrel 10. A stopper 17 is movably received in the barrel 10 and connected to a plunger 15 by a connection member 16 which is securely inserted into the stopper 17. A locking tip 180 is mounted to the engaging tube 12 and a needle cannula 181 is connected to the locking tip 180. Medicine in the interior 11 of the barrel 10 can be injected out from the needle cannula 181 by pushing the plunger 15 toward the engaging tube 12. A cap can be mounted to the locking tip to enclose the needle cannula 181. After being used, the syringe device has to be discarded and the needle cannula 181 together with the locking tip 180 are removed from the engaging tube 12. However, it is dangerous that when mounting the cap to enclose the needle cannula 181, the user could be cut by the needle cannula 181 and is therefore contaminated.

### SUMMARY OF THE INVENTION

In accordance with one aspect of the present invention, there is provided a syringe device and comprises a barrel having a first open end and a first flange extends radially inward from an inner periphery of the first open end. A finger flange extends radially outward from an outer periphery of a second open end of the barrel. A base member has an engaging tube extending from a first end thereof and an annular flange extends radially outward from a second end of the base member. A recess is defined in the second end of the base member and a groove is defined in an inner periphery of the recess. A first passage is defined through the engaging tube and communicates with the recess. The engaging tube extends through the first open end and the annular flange is engaged with the first flange.

A core member has a first insertion which is inserted in the recess of the base member. A plurality of first ribs extend from the first insertion of the core member and are engaged with the groove of the base member. A second insertion extends from a second end of the core member and has a plurality of second ribs extending radially therefrom. A path is defined radially in the core member and a second passage is defined in the first insertion. The path communicates with the second passage. A plunger has a central passage in which the core member is received. The first insertion extends from a first end of the plunger and a shoulder extends radially inward from the central passage and is located close to a second end of the plunger. The second ribs are engaged with the shoulder when the first end of the plunger is pushed to contact the second end of the base member.

The primary object of the present invention is to provide a syringe device wherein the needle cannula is retracted into the barrel by pulling the plunger and the plunger is limited to be pushed into the barrel again.

The present invention will become more obvious from the following description when taken in connection with the accompanying drawings which show, for purposes of illustration only, several preferred embodiments in accordance with the present invention.

### BRIEF DESCRIPTION OF THE DRAWINGS

FIG. 1 is a cross sectional view to show a conventional syringe device;

FIG. 2 is an exploded view to show a syringe device of the present invention;

FIG. 3 is a cross sectional view to show that the plunger is pulled away from the barrel of the syringe device of the present invention;

FIG. 4 is a cross sectional view to show that the plunger is pulled to a position where the plunger is stopped by an annular ridge in the barrel of the syringe device of the present invention;

FIG. 5 is a cross sectional view to show that the plunger is pushed to an extreme position in the barrel of the syringe device of the present invention;

FIG. 6 is a cross sectional view to show that the plunger is pulled and the base member is retracted in the barrel of the syringe device of the present invention;

FIG. 7 is a cross sectional view to show that the two flexible protrusions on the plunger are blocked between the barrel and the plunger to prevent the plunger from being pushed into the barrel again;

FIG. 8 is an exploded view to show another embodiment of the syringe device of the present invention;

FIG. 9 is a cross sectional view to show that the plunger in FIG. 8 is pulled away from the barrel of the syringe device of the present invention;

FIG. 10 is a cross sectional view to show that the plunger in FIG. 8 is pulled to a position where the plunger is stopped by an annular ridge in the barrel of the syringe device of the present invention;

FIG. 11 is a cross sectional view to show that the plunger in FIG. 8 is pushed to an extreme position in the barrel of the syringe device of the present invention;

FIG. 12 is a cross sectional view to show that the plunger in FIG. 8 is to be pulled and the flexible plate is disengaged from the hook on the plunger,

FIG. 13 is a cross sectional view to show that the plunger in FIG. 8 is pulled and the base member is retracted in the barrel of the syringe device of the present invention;

FIG. 14 is a cross sectional view to show that the two flexible protrusions on the plunger in FIG. 8 are blocked between the barrel and the plunger to prevent the plunger from being pushed into the barrel again, and

FIG. 15 is an exploded view to show yet another embodiment of the syringe device of the present invention.

### DETAILED DESCRIPTION OF THE PREFERRED EMBODIMENTS

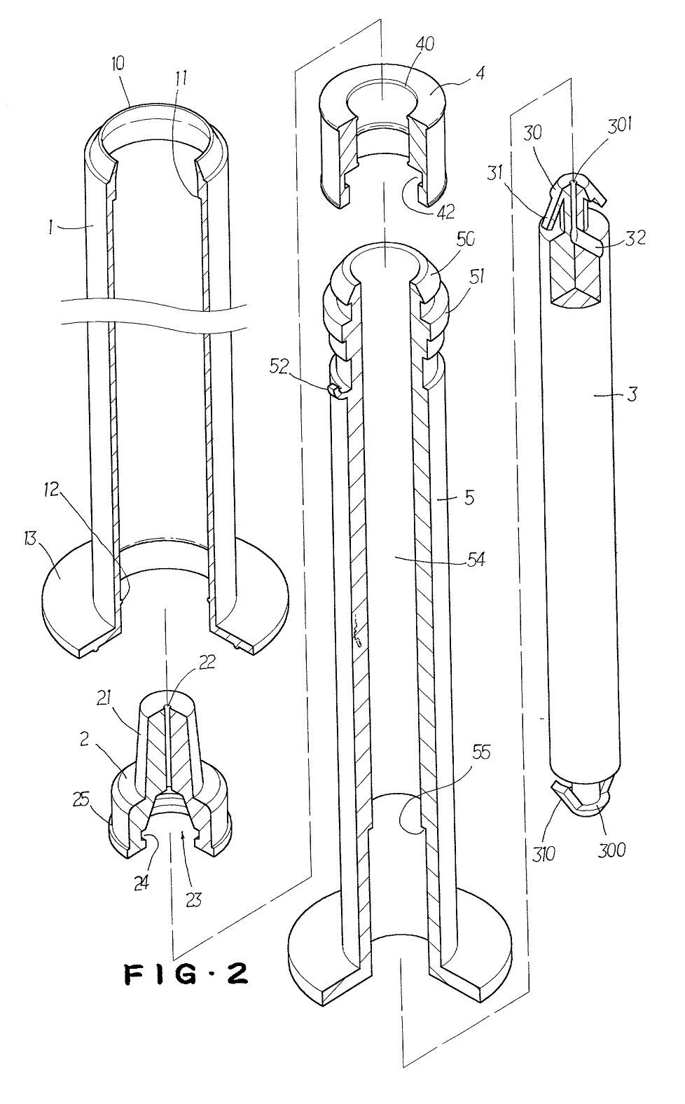

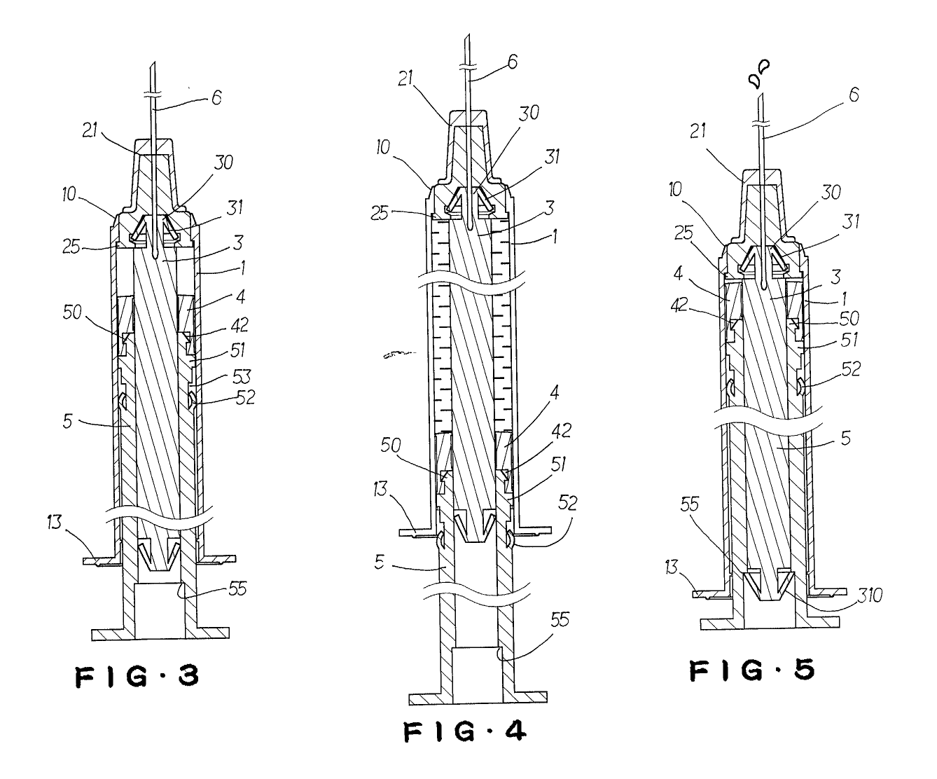

Referring to FIGS. 2 and 3, the syringe device of the present invention comprises a barrel 1 having a first open end 10 and a first flange 11 extending radially inward from an inner periphery of the first open end 10, a finger flange 13 extending radially outward from an outer periphery of a second open end of the barrel 1. An annular ridge 12 extends from the inner periphery of the second open end of the barrel 1.

A base member 2 has an engaging tube 21 extending from a first end thereof and an annular flange 25 extends radially outward from a second end of the base member 2. A recess 23 is defined in the second end of the base member 2 and a groove 24 is defined in an inner periphery of the recess 23. A first passage 22 is defined through the engaging tube 21 and communicates with the recess 23. The engaging tube 21 extends through the first open end 10 and the annular flange 25 engaged with the first flange 11. A needle cannula 6 is engaged with the first passage 22.

A core member 3 has a first insertion 30 extending from a first end of the core member 3 and the first insertion 30 is inserted in the recess 23 of the base member 2. A plurality of first ribs 31 extend from the first insertion 30 of the core member 3 and are engaged with the groove 24 of the base member 2 so that the base member 2 and the core member 3 are connected with each other. A second insertion 300 extends from a second end of the core member 3 and a plurality of second ribs 310 extend radially from the second insertion 300 of the core member 3. A path 32 is defined radially in the core member 3 and a second passage 301 is defined in the first insertion 30. The path 32 communicates with the second passage 301.

A plunger 5 has a central passage 54 in which the core member 3 is received. The first insertion 30 extends from a first end of the plunger 5 and a shoulder 55 extends radially inward from the central passage 54 and is located close to a second end of the plunger 5. An engaging flange 50 extends radially outward from the first end of the plunger 5 and a seal member 4 is mounted to the first end of the plunger 5. The seal member 4 has a groove 42 defined in an inner periphery thereof so that the engaging flange 50 is engaged with the groove 42 of the seal member 4. The seal member 4 has a hole 40 through which the core member 3 extends. The seal member 4 snugly and slidably contacts the inner periphery of the barrel 1 so that the medicine received in the space between the inner periphery of the barrel 1 and the seal member 4 can be pushed into the path 32, the second passage 301 and is ejected out from the needle cannula 6 when pushing the plunger 5 toward the base member 2. A limit flange 51 extends radially outward from the first end of the plunger 5. When sucking medicine from the needle cannula 6 into the barrel 1 by pulling the plunger 5, the limit flange 51 is limited from pulling out from the second open end of the barrel 1 by the annular ridge 12 as shown in FIG. 4. Two flexible protrusions 52 extend radially outward from the plunger 5 and are located close to the limit flange 51. The two flexible protrusions 52 contact on the inner periphery of the barrel 1 so that the plunger 5 is allowed to be moved in the barrel 1. When pushing the plunger 5 to eject the medicine, the first end of the plunger 5 is pushed to contact the second end of the base member 2, and at this position, the second ribs 310 are engaged with the shoulder 55 of the plunger 5 as shown in FIG. 5.

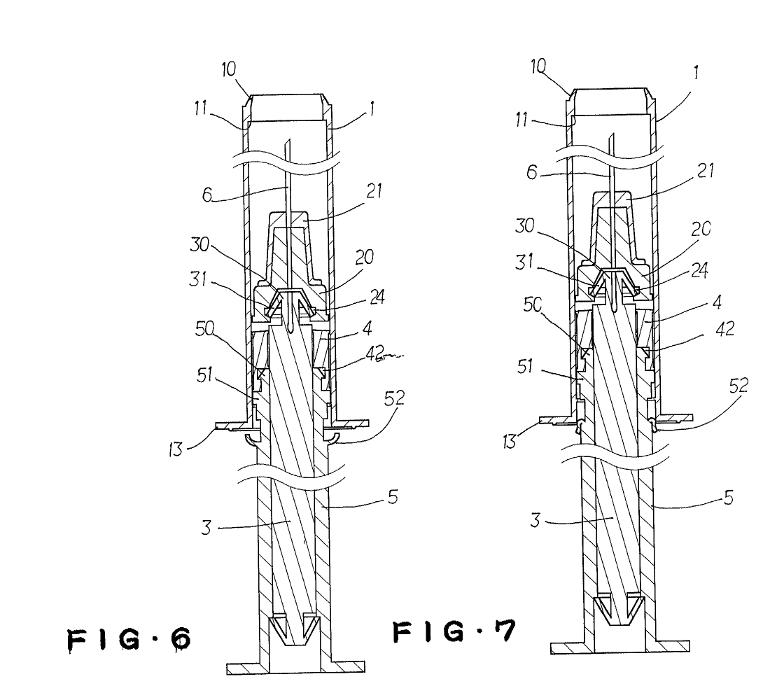

A shown in FIG. 6, after the medicine is completely ejected, the plunger 5 is pulled again, because the base member 2 is connected to the plunger 5, the base member 2 together with the needle cannula 6 are pulled into the barrel 1. The two flexible protrusions 52 are moved out from the second open end of the barrel 1 with the movement of the plunger 5. A distance between two respective distal ends of the two flexible protrusions 52 are larger than an inner diameter of the inner periphery of the second open end of the barrel 1. As shown in FIG. 7, the flexible protrusions 52 expand when they are disengaged from the second open end of the barrel 1 so that when the plunger 5 is pushed again by whatever reason, the two flexible protrusions 52 are blocked between the barrel 1 and the plunger 5 to prevent the plunger from being pushed into the barrel 1 again.

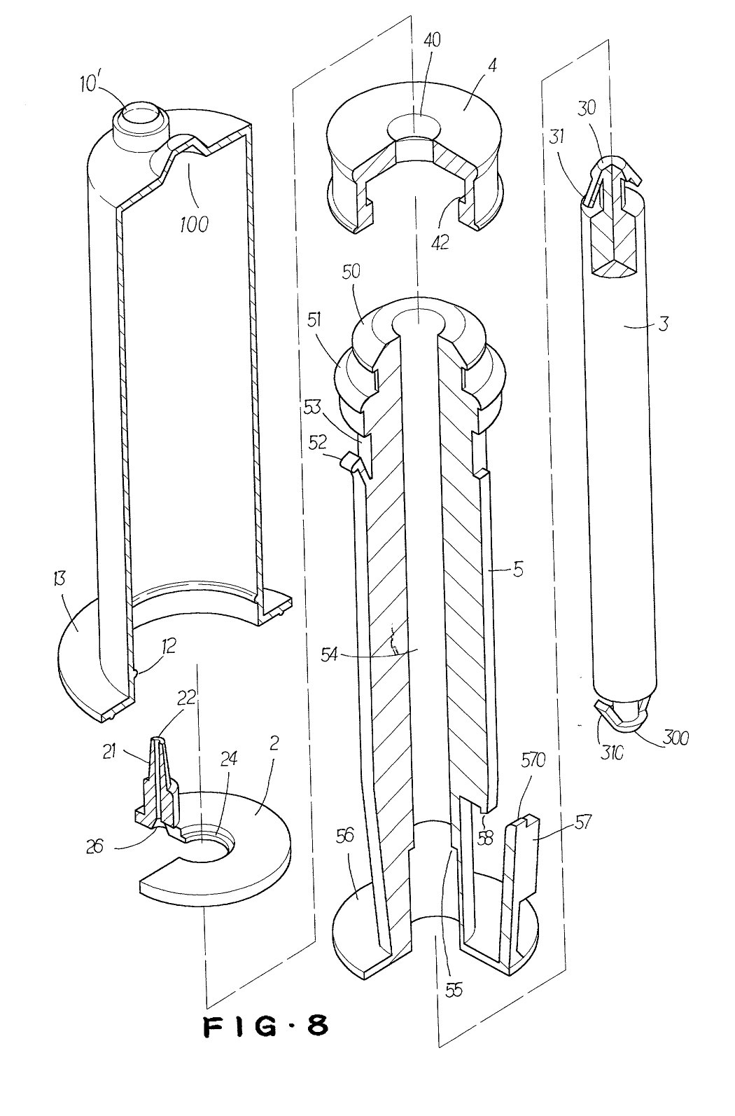

Referring to FIGS. 8 to 10, another embodiment of the syringe device includes a barrel 1 having an opening 10′ defined eccentrically through a first end of the barrel 1 and a concavity 100 is defined in an inside of the first end of the barrel 1. A finger flange 13 extends radially outward from an outer periphery of a second open end of the barrel 1. The barrel 1 has an annular ridge 12 extending from the inner periphery of the second open end. A base member 2 includes a ring and an engaging tube 21 extends from a first surface of the ring. A first passage 22 is defined through the engaging tube 21 and a groove 26 is defined in a second surface of the ring and communicates with the first passage 22. An annular flange 24 extends radially inward from a periphery of a central hole of the ring. The engaging tube 21 extends through the opening 10′.

A core member 3 has a first insertion 30 extending from a first end of the core member 3 and the first insertion 30 extends through the central hole of the ring and is inserted in the concavity 100 of the barrel 1. A plurality of first ribs 31 extend from the first insertion 30 of the core member 3 and are engaged with the annular flange 24 of the ring so that the core member 3 is connected to the base member 2. A second insertion 300 extends from a second end of the core member 3 and a plurality of second ribs 310 extend radially from the second insertion 300 of the core member 3.

A plunger 5 has a central passage 54 in which the core member 3 is received. The first insertion 30 extends from a first end of the plunger 5 and a shoulder 55 extends radially inward from the central passage 54 and is located close to a second end of the plunger 5. An engaging flange 50 extends radially outward from the first end of the plunger 5 and a seal member 4 is mounted to the first end of the plunger 5. The seal member 4 has a groove 42 defined in an inner periphery thereof so that the engaging flange 50 is engaged with the groove 42 of the seal member 4. The seal member 4 has a hole 40 through which the core member 3 extends. A thumb rest 56 extends radially outward from a second end of the plunger 5 and a flexible plate 57 extends from the thumb rest 56. A hook 58 is defined in an outer periphery of the plunger 5 and engagable with a tip portion 570 on the flexible plate 57. When the flexible plate 57 is not pushed inward to engage with the hook 58, the flexible plate 57 will contact the finger flange 13 and this limits the seal member 4 not to contact the base member 2 as shown in FIG. 9.

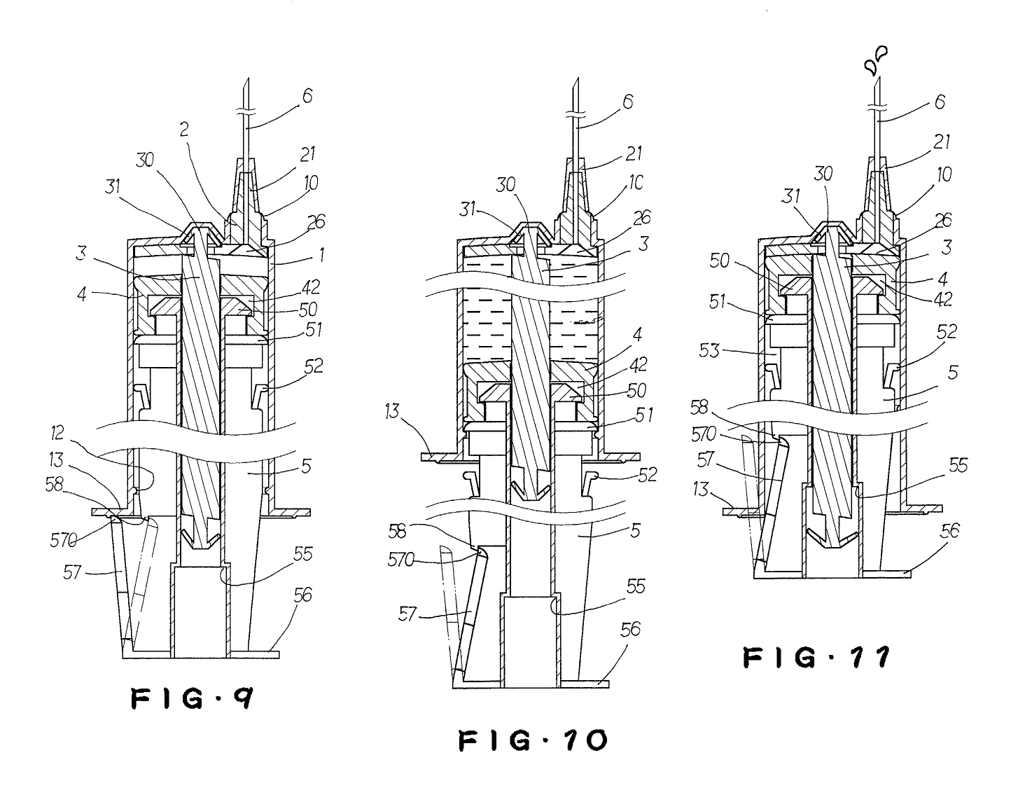

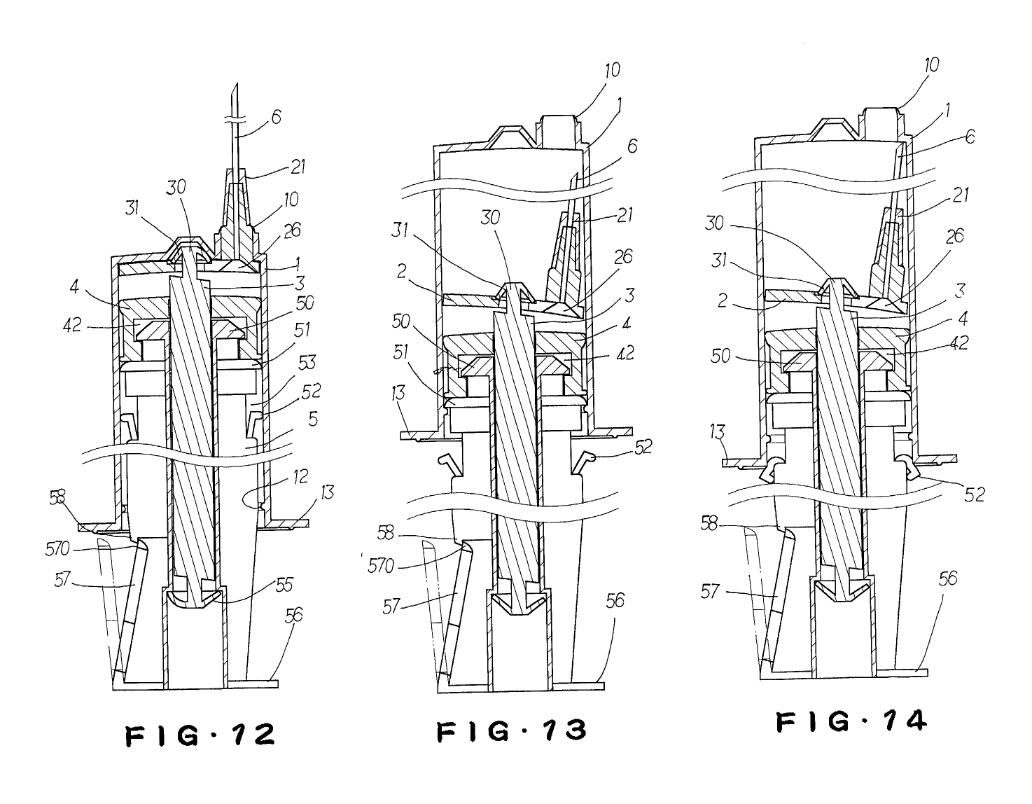

A limit flange 51 extends radially outward from the first end of the plunger 5 so that the limit flange 51 is limited from pulling out from the second open end of the barrel 1 by the annular ridge 12 as shown in FIG. 10 when the plunger 5 is pulled to suck medicine in the barrel 1. As shown in FIG. 11, when proceeding injection, the plunger 5 is pushed till the seal member 4 contacts the base member 2. The medicine is forced into the first passage 22 via the groove 26. When the seal member 4 contacts the base member 2, the second ribs 310 are engaged with the shoulder 55 when the first end of the plunger 5 is pushed to contact the second surface of the base member 2. When pulling the plunger 5 after the medicine is ejected, as shown in FIG. 12, the core member 3 is pulled together with the plunger 5 and the base member 2 together with the needle cannula 6 are pulled in the barrel 1 as shown in FIG. 13.

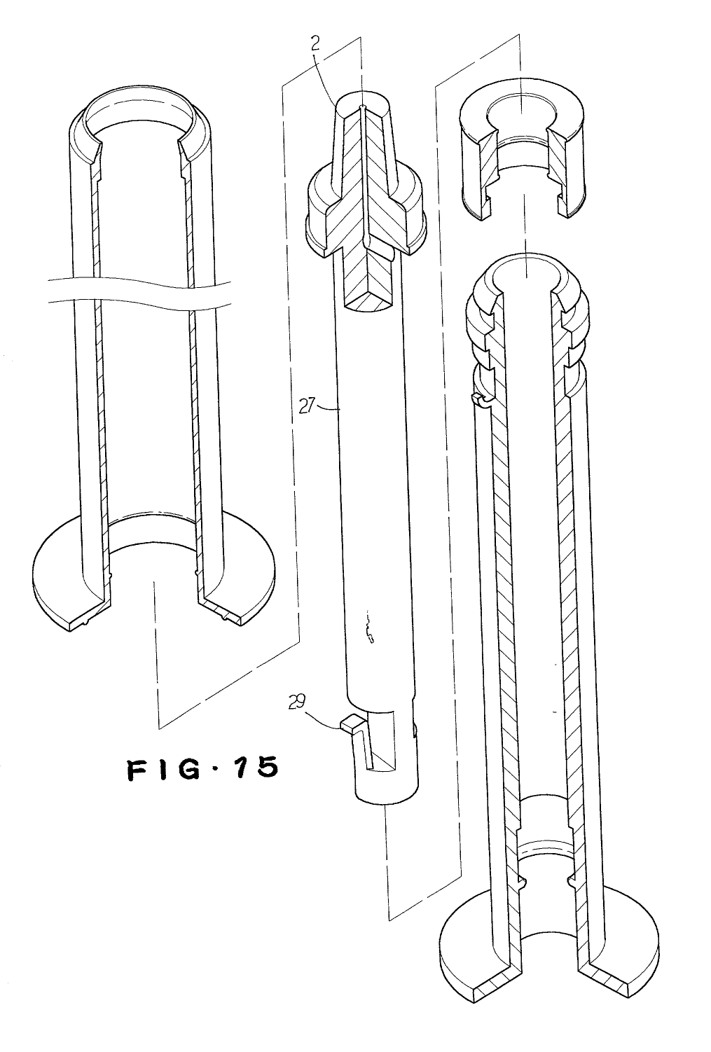

Two flexible protrusions 52 extend radially outward from the plunger 5 and are located close to the limit flange 51. A distance between two respective distal ends of the two flexible protrusions 52 is larger than an inner diameter of the inner periphery of the second open end of the barrel 1, so that when the plunger 5 is pulled to a position where the flexible protrusions 52 are located outside of the second open end of the barrel 1, the flexible protrusions 52 expand as shown in FIG. 13. Referring to FIG. 14, when the plunger 5 is pushed again, the two flexible protrusions 52 are blocked between the barrel 1 and the plunger 5 to prevent the plunger from being pushed into the barrel 1 again.

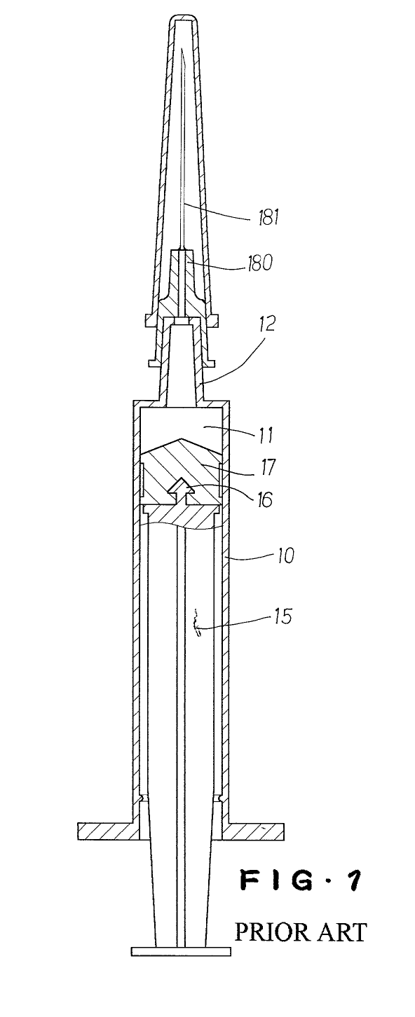

FIG. 15 shows yet another embodiment of the syringe device wherein the base member 2 and the core member 27 made to be a one-piece member, and the base member 2 is connected to a first end of the core member 27. A flexible rib 29 extends from a second end of the core member 27. This embodiment is similar to the syringe device as shown in FIG. 1.

While we have shown and described the embodiments in accordance with the present invention, it should be clear to those skilled in the art that further embodiments may be made without departing from the scope of the present invention.

What is claimed is:

1. A syringe device comprising:
a barrel having a first open end and a first flange extending radially inward from an inner periphery of said first open end, a finger flange extending radially outward from an outer periphery of a second open end of said barrel;
a base member having an engaging tube extending from a first end thereof and an annular flange extending radially outward from a second end of said base member, a recess defined in said second end of said base member and a groove defined in an inner periphery of said recess, a first passage defined through said engaging tube and communicating with said recess, said engaging tube extending through said first open end and said annular flange engaged with said first flange;
a core member having a first insertion extending from a first end of said core member and said first insertion inserted in said recess of said base member, a plurality of first ribs extending from said first insertion of said core member and engaged with said groove of said base member, a second insertion extending from a second end of said core member and a plurality of second ribs extending radially from said second insertion of said core member, a path defined radially in said core member and a second passage defined in said first insertion, said path communicating with said second passage, and
a plunger having a central passage in which said core member is received, said first insertion extending from a first end of said plunger and a shoulder extending radially inward from said central passage and located close to a second end of said plunger, said second ribs engaged with said shoulder when said first end of said plunger is pushed to contact said second end of said base member.

2. The syringe device as claimed in claim 1 further comprising an engaging flange extending radially outward from said first end of said plunger, a seal member mounted to said first end of said plunger and having a groove defined in an inner periphery thereof said engaging flange engaged with said groove of said seal member, said seal member having a hole through which said core member extends.

3. The syringe device as claimed in claim 1 further comprising a limit flange extending radially outward from said first end of said plunger, said barrel having an annular ridge extending from said inner periphery of said second open end, said limit flange being limited from pulling out from the second open end of said barrel by said annular ridge.

4. The syringe device as claimed in claim 3 further comprising two flexible protrusions extending radially outward from said plunger and located close to said limit flange, a distance between two respective distal ends of said two flexible protrusions being larger than an inner diameter of said inner periphery of said second open end of said barrel.

5. A syringe device comprising:
a barrel having an opening defined eccentrically through a first end of said barrel and a concavity defined in an inside of said first end of said barrel, a finger flange extending radially outward from an outer periphery of a second open end of said barrel;
a base member including a ring and an engaging tube extending from a first surface of said ring, a first passage defined through said engaging tube, a groove defined in a second surface of said ring and communicating with said first passage, an annular flange extending radially inward from a periphery of a central hole of said ring, said engaging tube extending through said opening;
a core member having a first insertion extending from a first end of said core member and said first insertion extending through said central hole of said ring and inserted in said concavity of said barrel, a plurality of first ribs extending from said first insertion of said core member and engaged with said annular flange of said ring, a second insertion extending from a second end of said core member and a plurality of second ribs extending radially from said second insertion of said core member, and
a plunger having a central passage in which said core member is received, said first insertion extending from a first end of said plunger and a shoulder extending radially inward from said central passage and located close to a second end of said plunger, said second ribs engaged with said shoulder when said first end of said plunger is pushed to contact said second surface of said base member.

6. The syringe device as claimed in claim 5 further comprising an engaging flange extending radially outward from said first end of said plunger, a seal member mounted to said first end of said plunger and having a groove defined in an inner periphery thereof, said engaging flange engaged with said groove of said seal member, said seal member having a hole through which said core member extends.

7. The syringe device as claimed in claim 5 further comprising a thumb rest extending radially outward from a second end of said plunger and a flexible plate extending from said thumb rest, a hook defined in an outer periphery of said plunger and engagable with said flexible plate.

8. The syringe device as claimed in claim 5 further comprising a limit flange extending radially outward from said first end of said plunger, said barrel having an annular ridge extending from said inner periphery of said second open end, said limit flange being limited from pulling out from the second open end of said barrel by said annular ridge.

9. The syringe device as claimed in claim 8 further comprising two flexible protrusions extending radially outward from said plunger and located close to said limit flange, a distance between two respective distal ends of said two flexible protrusions being larger than an inner diameter of said inner periphery of said second open end of said barrel.
